(19) United States
(12) Patent Application Publication
(10) Pub. No.: US 2025/0281698 A1
(43) Pub. Date: Sep. 11, 2025

(54) HYDROGEL AGITATION AND INJECTION ASSEMBLIES AND SYSTEMS

(71) Applicant: Boston Scientific Scimed, Inc., Maple Grove, MN (US)

(72) Inventor: Patrick Nicolas, Mansfield, MA (US)

(73) Assignee: Boston Scientific Scimed, Inc., Maple Grove, MN (US)

(21) Appl. No.: 19/069,741

(22) Filed: Mar. 4, 2025

Related U.S. Application Data
(60) Provisional application No. 63/561,440, filed on Mar. 5, 2024.

(51) Int. Cl. A61M 5/315

(52) U.S. Cl. CPC A61M 5/31596; A61M 2205/0288

(57) ABSTRACT

In some aspects, the present disclosure pertains to a hydrogel agitation and injection assembly, the assembly comprising a delivery device providing a barrel configured as a reservoir for an injectable hydrogel; and a shuttle disposed in the reservoir, where the shuttle is configured at least to translate along a longitudinal axis of the delivery device responsive to an application of a magnetic field to the shuttle, and where the shuttle includes an aperture extending entirely through the shuttle along the longitudinal axis of the delivery device.

### CROSS-REFERENCE TO RELATED APPLICATIONS

This application claims the benefit of U.S. Provisional Patent Application Ser. No. 63/561,440 filed on Mar. 5, 2024, the disclosure of which is incorporated herein by reference.

### FIELD

The present disclosure relates to hydrogel agitation and injection assemblies and systems. Hydrogels are useful, for example, in various medical applications.

### BACKGROUND

Hydrogels with shear-thinning properties are known which undergo a reversible gel-sol transition upon the application of shear stress. J. Pushpamalar, et al., “Development of a polysaccharide-based hydrogel drug delivery system (DDS): An update.” Gels, 2021, 7 (4), p.153. Shear-thinning hydrogels are increasingly used in drug delivery systems as a result of their ability to conform to the shape of an injection cavity, which maximizes contact with targeted tissue for localized drug delivery. Id. Hydrogels with shear-thinning properties have been reported to provide smooth injection without injection needle clogging, with the hydrogels returning to their original properties once mechanical load (shear stress) is removed. M. H. Chen, et al., “Methods to assess shear-thinning hydrogels for application as injectable biomaterials.” ACS Biomater. Sci. Eng. 2017, 3, 3146-3160. Hydrogels assembled by physical crosslinking of polyvinyl alcohol (PVA) and polyvinyl pyrrolidone (PVP) and containing doxorubicin have been noted to be able to deform under high shear and subsequently retain their original shape upon the removal of the high shear, demonstrating both shear-thinning and self-healing properties. N. K. Prasad, et al., “Discerning the self-healing, shear-thinning characteristics and therapeutic efficacy of hydrogel drug carriers migrating through constricted microchannel resembling blood microcapillary,” Colloids Surf. A Physiochem. Eng. Asp. 2021, 626, 127070. FIG. 1 on page 5 of J. Pushpamalar, et al., schematically illustrates the shear-thinning and self-healing properties of a doxorubicin-loaded poly(vinyl alcohol)/poly(vinyl pyrrolidone) hydrogel. A shear-thinning hydrogel containing gelatin and laponite for localized drug delivery and further containing chitosan and poly(N-isopropylacrylamide-co-acrylic acid) particles to render the hydrogel pH-responsive has also been reported. S. Gharaie, et al., “Smart shear-thinning hydrogels as injectable drug delivery systems.” Polymers, 2018, 10 (12), p. 1317.

### SUMMARY

The present disclosure provides hydrogel agitation and injection assemblies and systems with unique configurations as well as various novel applications for agitation and delivery of hydrogels (e.g., shear-thinning hydrogels) beyond drug delivery.

Potential benefits associated with the present disclosure include mitigation of any issues (e.g., the unintended introduction of air into a hydrogel) associated with previous hydrogel agitation approaches. For instance, unlike previous approaches which utilize two fluidically coupled syringes to agitate a hydrogel (e.g., by moving the hydrogel between a reservoir in a first syringe and a reservoir in a second syringe), the present disclosure effectively and efficiently agitates an injectable hydrogel without introduction of air into the injectable hydrogel.

Additional potential benefits associated with the present disclosure include one or more of the following: hydrogels are provided that can be injected smoothly by a health care provider, the health care provider can pause injection without risk of needle clogging during product injection, that mitigate or eliminate any imaging issues associated with the presence of air in a hydrogel, and a single syringe can be used for agitation and injection.

In some aspects, the present disclosure provides a hydrogel agitation and injection assembly, the assembly comprising: a delivery device providing a barrel configured as reservoir for an injectable hydrogel; and a shuttle disposed in the reservoir, wherein the shuttle is configured at least to translate along a longitudinal axis of the delivery device responsive to an application of a magnetic field to the shuttle, and wherein the shuttle includes an aperture extending entirely through the shuttle along the longitudinal axis of the delivery device.

In some embodiments, which can be used in conjunction with the above aspects, an outside diameter of the shuttle is equal to or less than an inside diameter of the barrel.

In some embodiments, which can be used in conjunction with the above aspects and embodiments, the shuttle is substantially cylindrical.

In some embodiments, which can be used in conjunction with the above aspects and embodiments, the shuttle is formed of a ferromagnetic material, wherein the shuttle includes a permanent magnet, or both.

In some embodiments, which can be used in conjunction with the above aspects and embodiments, the shuttle includes a plurality of permanent magnets including a first permanent magnet and a second permanent magnet.

In some embodiments, which can be used in conjunction with the above aspects and embodiments, the first permanent magnet is disposed at a first location, and wherein the second permanent magnet is disposed at a second location that is spaced a distance away from the first location.

In some embodiments, which can be used in conjunction with the above aspects and embodiments, the first location is located on a first side of the shuttle and wherein the second location is located on a second side of the shuttle that is opposite the first side.

In some embodiments, which can be used in conjunction with the above aspects and embodiments, the plurality of permanent magnets includes neodymium magnets, ceramic magnets, samarium cobalt magnets, or any combination thereof.

In some embodiments, which can be used in conjunction with the above aspects and embodiments, the shuttle includes a ferromagnetic material and has an absence of a permanent magnet.

In some embodiments, which can be used in conjunction with the above aspects and embodiments, the shuttle is configured to rotate about the longitudinal axis of the delivery device.

In some embodiments, which can be used in conjunction with the above aspects and embodiments, the aperture is an elongate aperture.

In some embodiments, which can be used in conjunction with the above aspects and embodiments, the elongate aperture is a linear elongate aperture.

In some embodiments, which can be used in conjunction with the above aspects and embodiments, the linear elongate aperture is a linear elongate slot or a linear elongate cylinder.

In some embodiments, which can be used in conjunction with the above aspects and embodiments, the shuttle includes radial fins.

In some embodiments, which can be used in conjunction with the above aspects and embodiments, the delivery device is a syringe including a barrel, a plunger, and a stopper, wherein the barrel is configured as the reservoir; and the syringe is a preloaded syringe that includes the hydrogel.

In some aspects the present disclosure provides an injectable hydrogel agitation and delivery system, the system comprising: syringe including a barrel, a plunger, and a stopper, wherein the barrel is configured as a reservoir for an injectable hydrogel; a shuttle disposed in the reservoir, wherein at least a portion of an outer surface of the shuttle is engaged with an inner surface of the barrel, and wherein the shuttle is configured to rotate about and translate along a longitudinal axis of the syringe to agitate an injectable hydrogel in the reservoir responsive to an application of a magnetic field to the shuttle, and wherein the shuttle includes an aperture extending entirely through the shuttle along the longitudinal axis of the syringe; and a magnetic activator including a magnet configured to emit the magnetic field to the shuttle.

In some embodiments, which can be used in conjunction with the above aspects and embodiments, the magnetic activator includes an arcuate region configured to mechanically couple to an outer surface of the barrel.

In some embodiments, which can be used in conjunction with the above aspects and embodiments the magnetic activator includes: a first end region extending from a first end of the arcuate region, wherein the first end region includes a first magnet disposed therein; and a second end region extending from a second end of the arcuate region, wherein the second end region includes a second magnet disposed therein.

In some aspects the present disclosure provides a kit comprising an injectable hydrogel comprising one or more types of polymeric hydrogen bond donors, one or more types of polymeric hydrogen bond acceptors, and water; syringe including a barrel, a plunger, and a stopper, wherein the barrel is configured as the reservoir for the injectable hydrogel; a shuttle disposed in the reservoir, wherein the shuttle is configured to rotate about and translate along a longitudinal axis of the syringe responsive to an application of a magnetic field to the shuttle, and wherein the shuttle includes an aperture extending entirely through the shuttle along the longitudinal axis of the syringe; and a magnetic activator including a magnet configured to emit the magnetic field to the shuttle, wherein the magnetic activator is configured to mechanically couple to an outer surface of the barrel of the syringe.

In some embodiments, which can be used in conjunction with the above aspects and embodiments, the syringe further comprises a preloaded syringe including the injectable hydrogel disposed in the reservoir.

The above and other aspects, embodiments, features and benefits of the present disclosure will be readily apparent from the following detailed description.

### BRIEF DESCRIPTION OF THE DRAWINGS

FIG. 1 schematically illustrates a catheter and a hydrogel agitation and injection assembly including a shuttle and a delivery device that is loaded with an injectable hydrogel, in accordance with the disclosure.

FIG. 2 schematically illustrates an example of system including a hydrogel agitation and injection assembly and a magnetic activator in accordance with the disclosure.

FIG. 3 schematically illustrates an exploded view of the system of FIG. 2.

FIG. 4 schematically illustrates an example of a shuttle.

FIG. 5 schematically illustrates another example of a shuttle.

FIG. 6 schematically illustrates an example of shuttle movement to agitate an injectable hydrogel in a reservoir responsive to an application of a magnetic field to the shuttle.

FIG. 7 schematically illustrates yet another example of a shuttle.

### DETAILED DESCRIPTION

This disclosure is now described with reference to an exemplary medical system that may be used in renal medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, the same or similar reference numbers will be used through the drawings to refer to the same or like parts.

The term “distal” refers to a portion farthest away from a user when introducing a device into a patient. By contrast, the term “proximal” refers to a portion closest to the user when placing the device into the patient. As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” Further, as used herein, the terms “about,” “approximately” and “substantially” indicate a range of values within +/−10% of a stated or implied value. Additionally, terms that indicate the geometric shape of a component/surface refer to exact and approximate shapes.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

Disclosed herein are the systems, devices, and methods herein that may be employed to agitate a fluid and/or a hydrogel in a reservoir of a medical delivery device. For instance, a hydrogel stored in a syringe for an extended time may exhibit settling of hydrogel particles and/or may exhibit a portion of a fluid (e.g., a carrier fluid) forming a separate phase (e.g., coming out of solution), thereby creating variation in a density of the hydrogel. Variations in the density of the hydrogel can cause issues with delivery (e.g., injection of the hydrogel) and/or may degrade or even negate the efficacy of the hydrogel. Accordingly, the systems, devices, and methods herein agitate a hydrogel stored in a reservoir of a delivery device such as a syringe. Yet, the systems, devices, and methods herein reduce a cost, a difficulty, and/or mitigate any issues (e.g., the unintended introduction of air into a hydrogel) that are associated with previous approaches for agitation of the hydrogel. For instance, unlike previous approaches which utilize two syringes that are coupled together to agitate a hydrogel therebetween, the present disclosure achieves agitation of an injectable hydrogel in the absence of an unintended introduction of air into the injectable hydrogel.

The carrier fluid in the injectable hydrogels of the present invention may be water. The water may be provided in the form of ultrapure water, water for injection, saline, phosphate buffered saline, or high-ion-content water.

In some embodiments, the injectable hydrogels contain between 0.25 weight percent (wt %) or less and 30 wt % or more water, for example, ranging anywhere from 0.25 to 0.5 to 1 to 2.5 to 5 to 10 to 20 to 30 wt %.

In some embodiments, the injectable hydrogels are shear-thinning and self-assembling injectable hydrogels. The shear-thinning properties of such hydrogels allow for efficient injectability, as the hydrogels exhibit viscous flow under shear. In some embodiments, the injectable hydrogels exhibit yielding behavior. For example, after being subjected to a threshold yield strain, the injectable hydrogels may exhibit sharp decreases in storage and loss moduli, which decreases in moduli are recovered at low strains upon cessation of shear. The self-assembling properties of such hydrogels (also referred to as self-healing properties) allows for re-formation and stabilization of the hydrogel when the shear stress is removed. As used herein, self-assembly and self-healing refer to the spontaneous formation of new bonds within a material after old bonds within the material are broken. As used herein, a hydrogel refers to a water-containing three-dimensional network of crosslinked polymers.

In various embodiments, the injectable hydrogels comprise (a) one or more types of hydrogen bond donors, (b) one or more types of hydrogen bond acceptors, and (c) water. Such hydrogels comprise hydrogen-bond-based crosslinks which dissociate when a shear stress is applied, and which spontaneously self-assemble when the shear stress is removed. Such disassociation may occur, for example, when a shear stress is applied during injection from a syringe. Upon dissociation of the hydrogen-bond-based crosslinks, the hydrogel becomes a viscous liquid that can be transported to a target site though a suitable delivery device, such as a tube (e.g. catheter/microcatheter) or a needle. Once delivered to the target site and the shear stress diminishes, the hydrogen bonds spontaneously re-associate (i.e., self-assemble), reforming the hydrogel at the target site. The transformation of the viscous liquid back into a hydrogel results in improved material retention and mechanical properties.

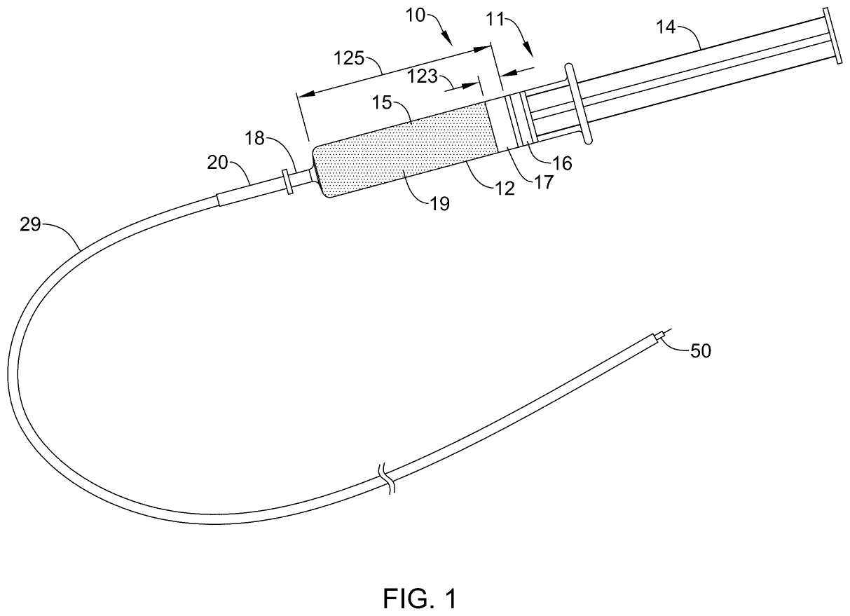

FIG. 1 schematically illustrates a catheter 29 and a hydrogel agitation and injection assembly 11 including a shuttle 17 and a delivery device 10 that is loaded with an injectable hydrogel, in accordance with an embodiment of the present disclosure. For instance, as illustrated in FIG. 1 the delivery device 10 may be a syringe configured to provide a reservoir 19 for an injectable hydrogel 15 and/or in which the shuttle 17 may be disposed, as is described herein. For example, the delivery device 10 may comprise a barrel 12, a plunger 14, and a stopper 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The barrel 12 may serve as the reservoir 19, containing an injectable hydrogel 15 for injection through the needle 50. The catheter 29 and/or the hydrogel agitation and injection assembly 11 may include additional components (e.g., seals such as O-rings, etc.).

In some embodiments, some or all of the components of the hydrogel agitation and injection assembly 11 may be configured to undergo sterilization (e.g., steam sterilization). In some embodiments, each of the components of the hydrogel agitation and injection assembly 11 may be configured to withstand steam sterilization. For instance, some or all of the components may be formed of materials that are suitable to withstand steam sterilization (e.g., retain their physical form and/or properties during and subsequent to undergoing steam sterilization). Examples of suitable materials (e.g., which retain their physical form) include glass, polycarbonate, polypropylene, rubber, and/or nylon, among other suitable materials.

In some embodiments, magnets may be employed in the shuttle 17 and/or in a magnetic activator, as described herein. Examples of suitable magnets (e.g., permanent magnets which remain magnetized during and after undergoing steam sterilization) include neodymium magnets, ceramic magnets, and/or samarium cobalt magnets, among other types of suitable magnets. For instance, a magnet (not illustrated in FIG. 1) in the shuttle 17 may be a neodymium magnet. Examples of suitable ferromagnetic materials include pure forms, alloys, or compounds of iron, cobalt, nickel, and/or rare-earth metals which exhibit ferromagnetism.

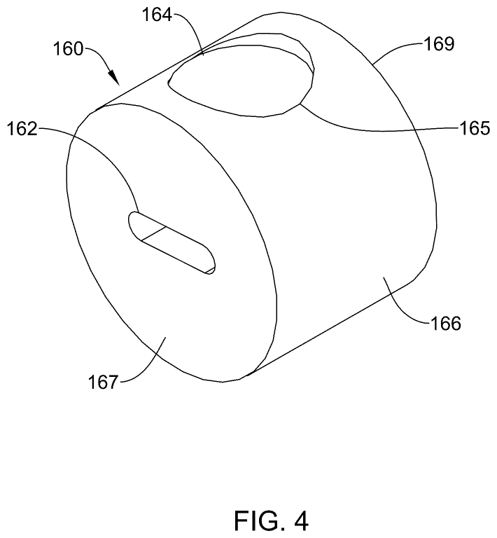

In some examples, the shuttle 17 can be a cylindrical shuttle with a circular or oval cross-section, for instance, as illustrated in FIG. 4. The shuttle 17 may be a cylindrical shuttle with a circular cross-section at any point along a longitudinal axis of the shuttle 17. The shuttle 17 can be configured to engage (e.g., form a fluidic seal) with an inner surface of the barrel 12. For instance, the shuttle 17 may be a substantially cylindrical shuttle (e.g., including or without one or more radially extending fins) that is shaped and sized to engaged with (e.g., contact and form a fluidic seal with) the adjacent portion of an inner surface of the barrel 12, as described herein. However, other shapes of the shuttle 17 are possible.

The shuttle 17 be configured to be disposed within a portion of the reservoir 19, as illustrated in FIG. 1. For instance, the shuttle 17 may have a length 123 (in a longitudinal direction) that is less than a length 125 of the reservoir 19 and may have a width e.g., an outside diameter (e.g., in a direction orthogonal to the longitudinal direction) that is equal to or is less than an inside diameter of the barrel 12. For instance, the shuttle 17 may have a length in a range from about 50 centimeters to about 1 centimeter, from about 40 centimeters to about 1 centimeter, from about 30 centimeters to about 1 centimeter, from about 20 centimeters to about 1 centimeter, from about 10 centimeters to about 1 centimeter, from about 5 centimeters to about 1 centimeter or from about 3 centimeters to about 1 centimeter. All sub-ranges and individual values from about 50 centimeters to about 1 centimeter are included.

The shuttle 17 may be configured with an outside diameter that is equal to or substantially equal to an inside diameter of the barrel 12. In this way, the shuttle 17 may be disposed inside the reservoir 19 and may be configured to have at least a portion of an outer surface of the shuttle 17 sealing engage (friction fit) with an inner surface of the barrel 12 and/or an inner surface of the reservoir 19. For instance, when the inner surface of the barrel 12 defines the reservoir 19, the shuttle 17 may be disposed inside the reservoir 19 and be configured to have at least a portion of an outer surface of the shuttle 17 friction fit with the inner surface of the barrel 12. In this way, the outer surface of the shuttle 17 may sealing engage with an adjacent portion of the inner surface of the barrel 12. When the outer surface of the shuttle 17 is sealing engaged with the inner surface of the barrel 12 fluid flow (e.g., such as the hydrogel 15 and/or carrier fluid included in the hydrogel 15) may not be permitted therebetween.

Having at least a portion of the outer surface of the shuttle 17 be sealing engaged with the adjacent portion of the inner surface of the barrel 12 may promote aspects herein such as promoting agitation of an injectable hydrogel in the reservoir 19 (e.g., as the shuttle 17 is translated along the reservoir 19). For instance, fluid (e.g., hydrogel 15 and/or carrier fluid of the hydrogel 15) in the reservoir 19 may, as the shuttle 17 translates along the longitudinal axis of a delivery device 10 (e.g., a syringe) may be forced through at least one aperture of the shuttle 17 thereby agitating the fluid in the reservoir, as is described herein. For instance, all or a predominant portion of the fluid in the reservoir 19 may be forced through at least one aperture of the shuttle 17 as the shuttle 17 translates longitudinally between a distal end of the reservoir 19 and a proximal end of the reservoir 19. Thus, the approaches herein can readily agitate an injectable hydrogel in the reservoir 19 without incurring the cost, difficulty, and/or issues (e.g., unintended introduction of air into the injectable hydrogel during agitation of the hydrogel between two coupled syringes) associated with previous approaches for agitation of the injectable hydrogel 15. When present, air may impart issues (e.g., storage issues and/or imaging issues once injected into a patient such as image scattering or other types of imaging defects).

In some embodiments, the shuttle 17 can be configured to move (e.g., translate) along a longitudinal axis of the delivery device responsive to an application of a magnetic field to the shuttle. In some embodiments, the shuttle 17 can be formed of a ferromagnetic material, can include a permanent magnet, or both.

In some embodiments, the shuttle 17 can be formed entirely of or can include a ferromagnetic material. For instance, the shuttle 17 can be formed entirely of or can include a ferromagnetic material and can have an absence of a permanent magnet. Having the shuttle 17 be configured with the absence of a permanent magnet can reduce a weight, complexity, and/or cost of the shuttle 17, and yet due to presence of the ferromagnetic material can permit the shuttle 17 to move responsive to the application of an externally applied magnetic field. However, in some instances, the shuttle 17 can be formed entirely of or can include a ferromagnetic material and can include at least one permanent magnet. Having the shuttle 17 can include at least one permanent magnet and also be formed of the ferromagnetic material can promote movement (e.g., translation and/or rotation) of the shuttle 17 responsive to the application of an externally applied magnetic field.

In various instances the shuttle 17 can include at least one permanent magnet. Having the shuttle 17 include at least one permanent magnet may promote aspects herein such as promoting the shuttle 17 to readily translate along the longitudinal axis of the delivery device responsive to application of the externally applied magnetic field (e.g., applied from a magnetic activator that is coupled to or proximate to the delivery device). The permanent magnet can be embedded in (e.g., does not protrude from an outer surface) the shuttle 17. Having the permanent magnet be embedded in the shuttle 17 may promote aspects herein, such as promoting movement of the shuttle along the longitudinal axis of the delivery device. For instance, the magnet can be disposed in a recess of a curved side surface of the shuttle 17, as is described herein. In some examples, the shuttle 17 can be formed of a non-ferromagnetic material (e.g., polycarbonate, rubber, nylon, polypropylene, etc.) and can include at least one permanent magnet embedded in the shuttle 17.

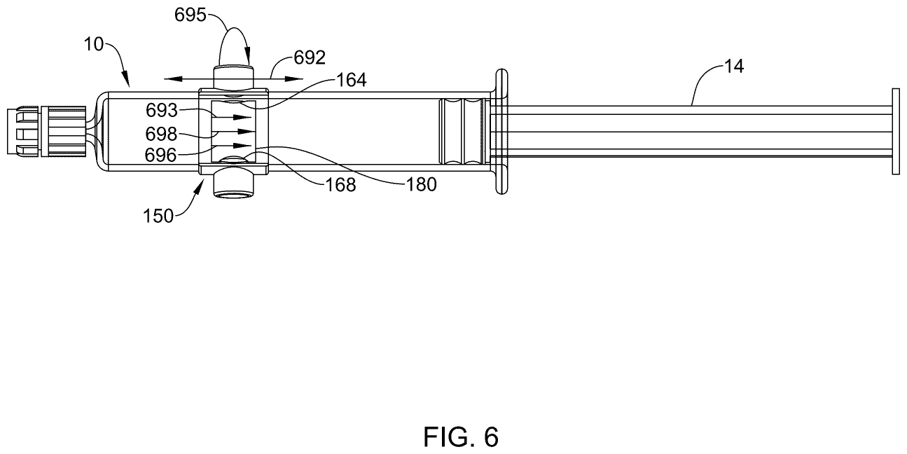

In some embodiments, the shuttle 17 can include plurality of permanent magnets including a first permanent magnet and a second permanent magnet. For instance, the shuttle 17 can include a first permanent magnet disposed at a first location and a second permanent magnet disposed at a second location that is spaced a distance away from the first location. Having the first magnet be disposed at a first location and a second permanent magnet be disposed at a second location that is spaced a distance away from the first location can promote aspects herein, such as promoting the translation and/or rotation of the shuttle 17 responsive to the application of an externally applied magnetic field. For example, the first location of the first magnet (e.g., magnet 164 as illustrated in FIG. 6) can be located on a first side of the shuttle 17 and the second location of the second magnet (e.g., magnet 168 as illustrated in FIG. 6) can be located on a second side of the shuttle 17 that is opposite the first side.

In some embodiments, the shuttle 17 can be configured to rotate about a longitudinal axis of the delivery device. For instance, the shuttle 17 can be configured to rotate about the longitudinal axis of the delivery device as the shuttle 17 is longitudinally translated along the longitudinal axis of the delivery device. For example, a shuttle configured to rotate about the longitudinal axis may include at least aperture, as described herein.

In some embodiments, the shuttle 17 includes at least one aperture (not illustrated in FIG. 1). The aperture can be an elongate aperture such as an elongate slot or an elongate hole, among other possibilities. The aperture can extend entirely through the shuttle 17 substantially along a longitudinal axis of the shuttle 17, as is described herein. For instance, the aperture can extend substantially along a longitudinal axis of the delivery device when the shuttle 17 is disposed in the delivery device.

In some examples, the aperture can be a linear (e.g., straight) elongate aperture. For instance, the aperture can be a linear aperture with respect to the longitudinal axis of the delivery device. For instance, some or all apertures in the shuttle 17 may be configured as linear elongate apertures. Employing linear elongate apparatus in the shuttle 17 can promote aspects herein such as providing a relatively short fluid flow path for the fluid in the reservoirs to pass through the shuttle 17.

In some embodiments, an aperture that extends through the shuttle 17 may be tapered or may include an inner surface that is textured and/or undulating (e.g., wavy). For instance, the shuttle 17 may include a first aperture that is tapered in a first direction (e.g., narrows from a proximal end to a distal end of the shuttle 17) and may include a second aperture that is tapered in second direction (e.g., narrows from a distal end to a proximal end of the shuttle. Having the aperture be tapered and/or include an inner surface that is textured and/or undulating may promote aspects herein such as promoting agitation of fluid (e.g., carrier fluid and hydrogel) responsive to the movement (e.g., the longitudinal translation) of the shuttle 17 within the reservoir.

In some embodiments, the aperture can be an angled elongate aperture that is configured at an angle relative to the longitudinal axis of the delivery device. For instance, some or all apertures in the shuttle 17 may be configured as angled elongate apertures. An angled aperture can be configured at an angle (relative to the longitudinal axis of the delivery device in which the shuttle is configured to be disposed) in a range from about 1 degree to about 60 degrees, in a range from about 1 degree to about 45 degrees, in a range from about 10 degree to about 60 degrees, in a range from about 10 degree to about 45 degrees, or can be in a range from about 30 degree to about 45 degrees. All individual values and sub-ranges from about 1 degree to about 60 degrees are included. Having at least one angled elongate aperture present in the shuttle 17 can promote aspects herein such as promoting rotation of the shuttle 17 about the longitudinal axis of the delivery device. For instance, fluid may exit the angled elongate apertures at an angle relative to the longitudinal axis of the delivery device, and thereby may impart a rotational force on the shuttle 17 as the shuttle is translated along the longitudinal axis of the delivery device. For instance, in some embodiments the shuttle 17 may include a plurality of angled elongate apertures such that the fluid exiting the plurality of angled elongate apertures experience helical agitation, thereby ensuring that the injectable hydrogel, once agitated by the shuttle 17, is uniform (e.g., has the same density and relative amounts of components throughout an entire volume of the injectable hydrogel). For instance, the carrier fluid may be evenly dispersed throughout an entire volume of the injectable hydrogel, once agitated by the shuttle 17.

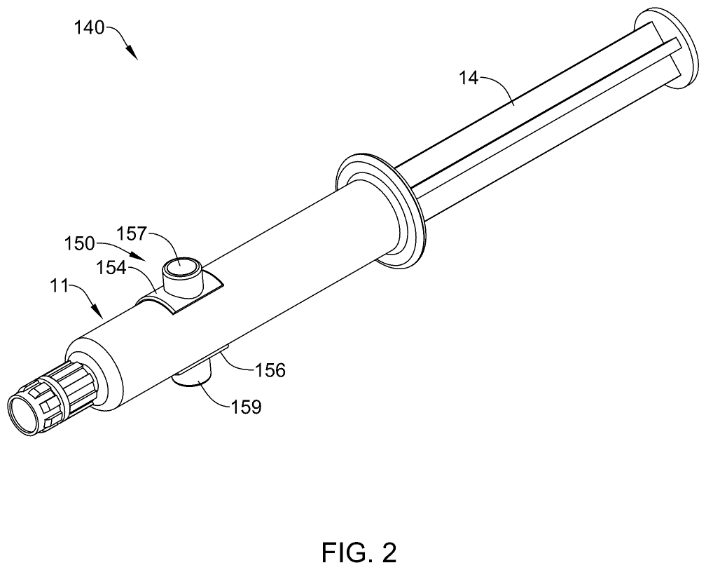

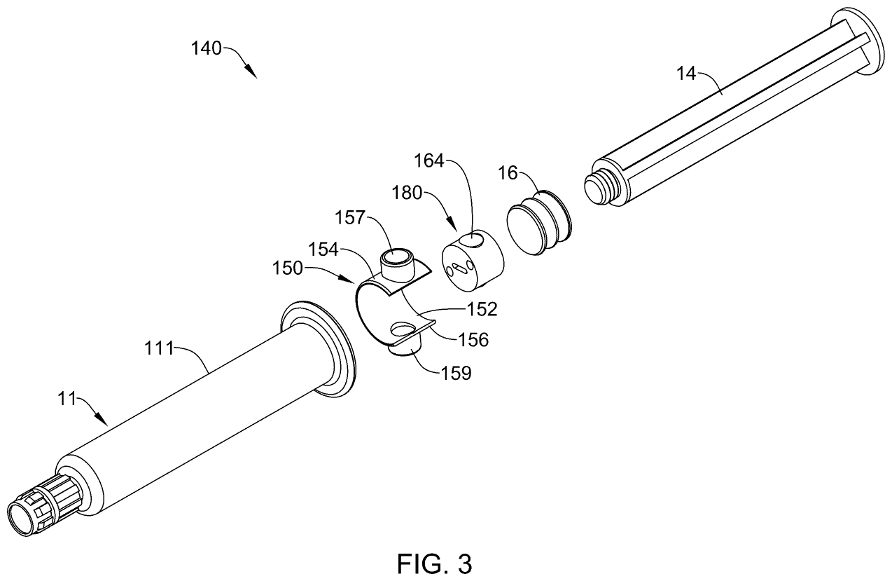

FIG. 2 schematically illustrates an example of system 140 including a hydrogel agitation and injection assembly 11 and a magnetic activator 150 in accordance with the disclosure, while FIG. 3 schematically illustrates an exploded view of the system 140. The hydrogel agitation and injection assembly 11 can be analogous to or similar to the hydrogel agitation and injection assembly 11 described with respect to FIG. 1.

The magnetic activator 150 can include an arcuate region 152 configured to mechanically couple the magnetic activator 150 to an outer surface 111 of the barrel. For instance, the arcuate region 152 may be shaped and sized (e.g., depending on the shape and size of a delivery device such as a syringe) to couple to or be in close proximity to the outer surface 111 of the delivery device at a location that is adjacent to a reservoir in the delivery device, as illustrated in FIG. 2. In this way, the magnetic activator 150 can be readily moved (e.g., translated and/or rotated) when coupled to or in close proximity to a shuttle that is disposed in a deliver device.

The magnetic activator 150 can include a first end region 154 extending from a first end of the arcuate region 152 and a second end region 156 extending from a second end of the arcuate region 152, as illustrated in FIG. 2. The first end region 154 and/or the second end region 156 may be configured as a handle (e.g., to permit a user to physically move the magnetic activator 150 relative to the delivery device such as a syringe). The first end region 154 and/or the second end region 156 may include a magnet. For instance, each of the first end region 154 and the second end region 156 may include a magnet (e.g., permanent) disposed therein, as illustrated in FIG. 2 and FIG. 3. For example, the first end region 154 can include a first permanent magnet 157 and the second end region 156 can include a second permanent magnet 159. Having a permanent magnet disposed in each of the first end region 154 and the second end region 156 may promote aspects herein such as promoting the simultaneous translation and rotation of the shuttle within a delivery device, thereby promoting agitation of a fluid (e.g., in injectable hydrogel) in a reservoir in which the shuttle is moving therein.

FIG. 4 schematically illustrates an example of a shuttle 160. As illustrated in FIG. 4, the shuttle 160 can include an individual elongate aperture 162 extending therethrough and include an individual permanent magnet 164. For instance, the individual elongate aperture 162 can extend entirely through the shuttle 160. The individual elongate aperture 162 can be a linear elongate aperture having a circular cross-section extending along at least a portion or along an entire length of the linear elongate aperture. However, the individual elongate aperture 162 can have a different shape or configuration such as those described herein.

The magnet 164 can be embedded within an opening 165 in the shuttle 160. For instance, as illustrated in FIG. 4, the shuttle 160 can be a cylindrical shuttle with the magnet 164 embedded in a curved side surface 166 of the cylindrical shuttle that extends between a first end 167 and a second end 169 of the cylindrical shuttle. The magnet 164 may therefore be positioned relatively proximate to an inner surface of a barrel of a syringe and thus may promote aspects herein such as causing the shuttle 160 to readily move (e.g., longitudinally translate) along the reservoir of the syringe in response to the application of an externally applied magnetic field by a magnetic activator. While FIG. 4 illustrates a shuttle including an individual elongate aperture 162 and an individual magnet 164, a quantity of apertures and/or magnets may be varied (e.g., increased), as described herein.

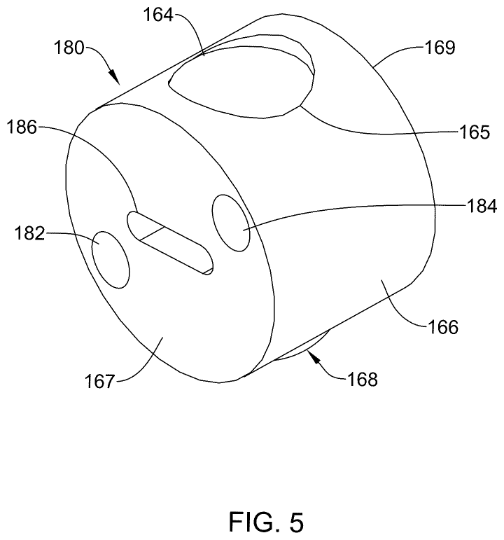

For instance, FIG. 5 schematically illustrates an example shuttle 180 including a plurality of apertures and a plurality of magnets. Similar to the shuttle 160 in FIG. 4, the shuttle 180 can be substantially cylindrical and can otherwise be configured to be disposed within a reservoir of a delivery device (e.g., a reservoir in a syringe).

As mentioned, the shuttle 180 can include a plurality of apertures in the form of a first aperture 182, a second aperture 184, and a third aperture 186. As illustrated in FIG. 5, the first aperture 182 and the second aperture 184 can be disposed between the third aperture 186 and a curved side surface 166 of the shuttle 180. The first aperture 182 and the second aperture 184 can be the same shape and size, while the third aperture 186 can be a different shape and size than the first aperture 182 and the second aperture 184. For instance, the first aperture 182 and the second aperture 184 can each be one or more linear elongate apertures such as linear elongate cylinders having a circular cross-section extending along a portion or an entire length thereof. The third aperture 186 can be linear elongate aperture such as a linear elongate slot having a slot-like cross-section extending along a portion or an entire length thereof. As illustrated in FIGS. 5-6, each of the first aperture 182, a second aperture 184, and a third aperture 186 can extend entirely through the shuttle 180. That is, each of the first aperture 182, the second aperture 184, and the third aperture 186 can extend between a first end 167 and a second end 169 of the respective shuttle 180.

While illustrated in FIGS. 5-6 as being linear elongate apertures (with respect to a longitudinal axis of the delivery device and a respective shuttle), the first aperture 182, a second aperture 184, and/or a third aperture 186 may be configured as an angled aperture. In some embodiments, at least two of the first aperture 182, the second aperture 184, and the third aperture 186 can be angled apertures that are configured to extend through the shuttle 180 at a non-zero angle with respect to a longitudinal axis of the shuttle 180. In some embodiments, each of the first aperture 182 and the second aperture 184 can be configured as angled apertures, while the third aperture 186 can be configured as a linear aperture (e.g., that extends coaxially with the longitudinal axis of a delivery device in which the shuttle 180 is disposed).

For instance, fluid in the reservoir that is proximate to the first end 167 or the second end 169 can be forced through each of the first aperture 182, a second aperture 184, and a third aperture 186 to the other of the first end 167 or the second end 169 as a shuttle is moved (e.g., is longitudinally translated) within the reservoir of the delivery device 10 (e.g., a syringe) thereby agitating the fluid in the reservoir. For example, as illustrated in FIG. 6 a shuttle such as the shuttle 180 may be longitudinally translated in a first direction 692 and may be rotated in a second direction 695 by virtue of application of an externally generated field applied from the magnetic activator 150 (e.g., which is analogous to the magnetic activator 150 illustrated in FIG. 2).

When the shuttle is rotated and translated, the fluid in a reservoir in which the shuttle is disposed can form at least one vortex and/or at least one fluid flow path that is present at an angle to another fluid flow path thereby promoting agitation of the fluid. For instance, as illustrated in FIG. 6, a first fluid flow path (represented as arrow 693) may extend through the first aperture (e.g., the first aperture 182 as illustrated in FIG. 5), a second fluid flow path (represented as arrow 696) may extend through the second aperture (e.g., the second aperture 184 as illustrated in FIG. 5), and the third fluid flow path (represented as arrow 698) may extend through the third aperture (e.g., the third aperture 186 (e.g., at least longitudinal translation) of the shuttle 180. For instance, the first fluid flow path 693 and the second fluid flow path 696 may be angled fluid flow paths with respect to the third fluid flow path 698. Having at least one of the fluid flow paths (e.g., the first and second fluid flow paths) can promote aspects herein such as promoting efficient and effective agitation of a fluid (e.g., an injectable hydrogel in the reservoir of a delivery device.

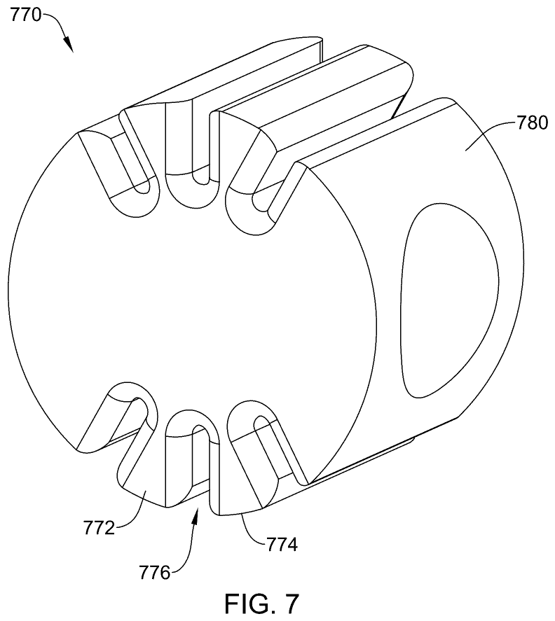

While the shuttle is described above as being a generally cylindrical shuttle, other shapes are possible. In some embodiments a shuttle can include one or more radial fins extending therefrom. In such instances, at least one aperture in a shuttle can be manifested as a space between adjacent radial fins. For instance, a shuttle 770 can include a plurality of radial fins including a first radial fin 772 and a second radial fin 774, as illustrated in FIG. 7. In such instances, an aperture in the form of a linear elongate slot 776 is located between the first radial fin 772 and the second radial fin 774, as illustrated in FIG. 7. In such embodiments, the distal tips of the radial fins and a curved side surface 780 of the shuttle 770 may be engaged with an inner surface of a barrel of a syringe when the shuttle is disposed in a reservoir defined by the barrel of the syringe.

In some embodiments, an injectable hydrogel is provided which can undergo disassembly (shear-thinning) during injection through a syringe, thereby providing smooth injection, and then re-assemble at a target site when the shear forces associated with the injection are removed. In addition, the shape of the hydrogel is transformed during injection and a new shape is established at the target injection site due to the self-assembling characteristics of the hydrogel. The ability of the viscous liquid to transform back into a hydrogel improves material retention and restores mechanical properties of the hydrogel. Such properties are useful, for example, in creating spacers for radiation and other cancer therapies and in injectable embolization applications.

In some embodiments, the injectable hydrogels further comprise one or more radiopaque atoms, which may be selected, for example, from Br, I, Bi, Ba, Gd, Ta, Zn, W and Au.

In various embodiments, the hydrogels of the present disclosure are visible under fluoroscopy. In various embodiments, the hydrogels have a radiopacity that is greater than 10 Hounsfield units (HU), beneficially anywhere ranging from 10 HU to 250 HU to 100 HU to 250 HU to 500 HU to 750 HU to 1000 HU to 2000 HU or more (in other words, ranging between any two of the preceding numerical values).

The injectable hydrogels herein may be formed using a variety of methods. The one or more types of hydrogen bond donors, the one or more types of hydrogen bond acceptors and the water may be mixed in any order. For example, the one or more types of hydrogen bond donors and the one or more types of hydrogen bond acceptors may be first mixed and then combined with the water. As another example, the one or more types of hydrogen bond donors and the water may be first mixed and then combined with the one or more types of hydrogen bond acceptors. As another example, the one or more types of hydrogen bond acceptors and the water may be first mixed and then combined with the one or more types of hydrogen bond donors. As another example, a first composition containing the one or more types of hydrogen bond donors and water may be formed, a second composition containing the one or more types of hydrogen bond acceptors and water may be formed, and the first and second compositions then combined. As yet another example, the one or more types of hydrogen bond acceptors, the one or more types of hydrogen bond donors and the water may be simultaneously mixed. Mixing may be performed by any suitable mixing technique, including, for example, centrifugal mixing, manual mixing, high shear dispersing, vacuum mixing, vortexing, and/or syringe-to-syringe mixing.

The compositions of the present disclosure may be sterilized using any suitable method such as heat (e.g., dry heat, moist heat, etc.), sterile filtration, supercritical CO₂, gamma-ray irradiation, x-ray irradiation or electron beam irradiation. The compositions of the present disclosure can be sterilized in hydrogel form or in powder form (to which a sterile liquid such as water, saline, etc. may be added). The compositions may be sterilized while inside a reservoir, such as a syringe barrel, vial, or ampule.

In some embodiments, the injectable hydrogels herein contain one or more agents in addition to the one or more types of hydrogen bond donors, the one or more types of hydrogen bond acceptors and the water. Examples of such additional agents include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antibodies, anti-cancer drugs, antiproliferative agents, anti-inflammatory agents, hyperplasia inhibiting agents, anti-restenosis agents, steroids, anti-allergic agents, hemostatic agents, smooth muscle cell inhibitors, antibiotics, antimicrobials, anti-fungal agents, analgesics, anesthetics, immunosuppressants, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, and STING (stimulator of interferon genes) agonists, among others.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd^{(III)}, Mn^{(II)}, Fe^{(III)} and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the injectable hydrogels, allowing for deep tissue imaging and device marking, for instance, NIR-sensitive nanoparticles such as gold nanoshells, carbon nanotubes (e.g., nanotubes derivatized with hydroxyl or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (e) imageable radioisotopes including ^{99m}Tc, ²⁰¹Th, ⁵¹Cr, ⁶⁷Ga, ⁶⁸Ga, ¹¹¹ In, ⁶⁴Cu, ⁸⁹Zr, ⁵⁹Fe, ⁴²K, ⁸²Rb, ²⁴Na, ⁴⁵Ti, ⁴⁴Sc, ⁵¹Cr and ¹⁷⁷Lu, among others, and (f) radiocontrast agents such as metallic particles, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block copolymers, etc.), among others, and pH adjusting agents including various buffer solutes.

In some embodiments, the injectable hydrogels may be stored and transported in a sterile form. The injectable hydrogels may be shipped, for example, in a syringe, catheter, vial, ampoule, or other container.

In various embodiments, kits are provided, which may include one or more delivery devices from injectable hydrogels as described herein as well other components such as a hydrogel agitation and injection assembly. For example, the kits may include a hydrogel agitation and injection assembly along with one or more delivery devices for delivering the injectable hydrogels to a subject such as syringes, catheters or tubing sets. In some embodiments, the kits may comprise an injectable hydrogel as described herein preloaded in a catheter and/or a syringe barrel and/or in a container such as a vial or ampule. Alternatively or in addition, kits may be provided that include one or more accessory devices such as guidewires. Alternatively or in addition, the kits may be provided that include one or more containers of liquid materials (e.g., contrast agent, sterile water for injection, physiological saline, phosphate buffer, etc.). Alternatively or in addition, the kits may further comprise an additional therapeutic agent, which may be selected, for example, from those described above, among others. Instructions, either as inserts or as labels, indicating quantities of the composition to be administered and/or guidelines for administration can also be included in the kits provided herein. In some embodiments, the instructions comprise instructions for performing one or more of the methods provided herein.

The injectable hydrogels described herein can be administered to patients for achieving a number of medical outcomes. The injectable hydrogels described herein can be administered by a variety of routes, depending upon the desired medical outcome. In some embodiments, the administering comprises injecting the injectable hydrogel. In some embodiments, the injectable hydrogels are administered by parenteral administration. Parenteral administration includes intravenous, intraarterial, subcutaneous, intraperitoneal, or intramuscular injection or infusion. In some embodiments, the parenteral administration is performed under image guidance. For instance, in some embodiments the administering comprises an image guided procedure where computed tomography, fluoroscopy or ultrasound imaging is used to deliver the composition. In some embodiments, the administering comprises injecting the injectable hydrogel into the vascular system of a subject. In some embodiments, the administering comprises injecting the injectable hydrogel into a tumor of the subject or the vasculature supplying a tumor of the subject. In some embodiments, the administering is performed using a catheter or a syringe.

The injectable hydrogels described herein can be visualized (e.g., within a mammal) using any appropriate method during and/or after administration. For example, imaging techniques such as ultrasound, computed tomography, magnetic resonance imaging, and/or fluoroscopy can be used to visualize the injectable hydrogels provided herein.

In some embodiments, the injectable hydrogels may be used in a variety of medical procedures, including the following, among others: a procedure to inject the injectable hydrogel into a feeder artery to embolize tissue, including benign tumors, malignant tumors and other abnormal tissue, a procedure to introduce the injectable hydrogel between a first tissue and a second tissue to space the first tissue from the second tissue, a procedure to implant a fiducial marker comprising the injectable hydrogel (e.g., in the form of blebs), a procedure to implant a tissue regeneration scaffold comprising the injectable hydrogel, a procedure to implant a tissue support comprising the injectable hydrogel, a procedure to implant a tissue bulking agent comprising the injectable hydrogel, a procedure to implant a therapeutic-agent-containing depot comprising the injectable hydrogel, a tissue augmentation procedure comprising implanting the injectable hydrogel, or a procedure to control bleeding.

The injectable hydrogels can be injected for tissue augmentation or regeneration, the injectable hydrogels can be injected as a filler or replacement for soft tissue, the injectable hydrogels can be injected to provide mechanical support for compromised tissue, the injectable hydrogels can be injected as a scaffold, and/or the injectable hydrogels can be injected as a carrier of therapeutic agents in the treatment of diseases and cancers and the repair and regeneration of tissue, among other uses.

The injectable hydrogels may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumors and polyps, visceral pleura injection for lung biopsy, kidney injection for type 2 diabetes and chronic kidney disease, renal cortex injection for chronic kidney disease from congenital anomalies of kidney and urinary tract, intra-vitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

The injectable hydrogels may be injected for the permanent or temporary occlusion of blood vessels, and thus may be useful for managing various diseases and conditions. For example, the injectable hydrogels may be used for the controlled, selective obliteration of the blood supply to benign and malignant tumors including treating solid tumors such as renal carcinoma, bone cancer, brain cancer, liver cancer, breast cancer, prostate cancer, benign prostatic hyperplasia, esophageal cancer, colon cancer, endometrial cancer, bladder cancer, cancer of the uterus, uterine fibroids (leiomyoma), cancer of the ovary, lung cancer, sarcoma, pancreatic cancer, and stomach cancer. The idea behind this treatment is that the flow of blood, which supplies nutrients to the tumor, will be blocked causing it to shrink. Embolization may be conducted as an enhancement to chemotherapy or radiation therapy. Treatment may be enhanced in the present disclosure by including a therapeutic agent (e.g., antineoplastic/antiproliferative/anti-miotic agent, toxin, ablation agent, etc.) in the injectable hydrogel.

Injectable hydrogels in accordance with the present disclosure may also be used to treat various other diseases, conditions and disorders, including treatment of the following: arteriovenous fistulas and malformations including, for example, aneurysms such as neurovascular and aortic aneurysms, pulmonary artery pseudoaneurysms, intracerebral arteriovenous fistula, cavernous sinus dural arteriovenous fistula and arterioportal fistula, varices, chronic venous insufficiency, varicocele, abscesses, pelvic congestion syndrome, gastrointestinal bleeding, renal bleeding, urinary bleeding, varicose bleeding, venous congestion disorder, hemorrhage, including uterine hemorrhage, and severe bleeding from the nose (epistaxis), as well as preoperative embolization (to reduce the amount of bleeding during a surgical procedure) and occlusion of saphenous vein side branches in a saphenous bypass graft procedure, among other uses. As elsewhere herein, treatment may be enhanced in the present disclosure by including a therapeutic agent in the particulate composition.

Injectable hydrogels in accordance with the present disclosure may be used further in tissue bulking applications, for example, as augmentative materials in the treatment of urinary incontinence, vesicourethral reflux, fecal incontinence, intrinsic sphincter deficiency (ISD) or gastro-esophageal reflux disease, or as augmentative materials for aesthetic improvement. For instance, a common method for treating patients with urinary incontinence is via periurethral or transperineal injection of a bulking material. In this regard, methods of injecting bulking agents commonly require the placement of a needle at a treatment region, for example, periurethrally or transperineally. The bulking agent is injected into a plurality of locations, assisted by visual aids, causing the urethral lining to coapt. In some cases, additional applications of bulking agent may be required. Treatment may be enhanced by including a therapeutic agent (e.g., proinflammatory agents, sclerosing agents, etc.) in the injectable hydrogel.

Injectable hydrogels in accordance with the present disclosure may be used in hemostasis, for example, by direct application to a bleeding site or injection into blood vessel leading to a bleeding site.

Injectable hydrogels in accordance with the present disclosure may be injected into a left atrial appendage during a left atrial appendage closure procedure. In some embodiments, the injectable hydrogels may be injected into the left atrial appendage after the introduction of a closure device such as the Watchman® left atrial appendage closure device available from the Boston Scientific Corporation.

Injectable hydrogels in accordance with the present disclosure may be used in the treatment of aneurisms. For example, the injectable hydrogels may be introduced into an aneurism, either alone or with an embolic device such as an embolic coil or a liquid embolic.

Although various embodiments are specifically illustrated and described herein, it will be appreciated that modifications and variations of the present disclosure are covered by the above teachings and are within the purview of any appended claims without departing from the spirit and intended scope of the present disclosure.

What is claimed is:

1. A hydrogel agitation and injection assembly, the assembly comprising:
a delivery device providing a barrel configured as a reservoir for an injectable hydrogel; and
a shuttle disposed in the reservoir, wherein the shuttle is configured at least to translate along a longitudinal axis of the delivery device responsive to an application of a magnetic field to the shuttle, and wherein the shuttle includes an aperture extending entirely through the shuttle along the longitudinal axis of the delivery device.

2. The assembly of claim 1, wherein an outside diameter of the shuttle is equal to or less than an inside diameter of the barrel.

3. The assembly of claim 1, wherein the shuttle is substantially cylindrical.

4. The assembly of claim 1, wherein the shuttle is formed of a ferromagnetic material, wherein the shuttle includes a permanent magnet, or both.

5. The assembly of claim 1, wherein the shuttle includes a plurality of permanent magnets including a first permanent magnet and a second permanent magnet.

6. The assembly of claim 5, wherein the first permanent magnet is disposed at a first location, and wherein the second permanent magnet is disposed at a second location that is spaced a distance away from the first location.

7. The assembly of claim 6, wherein the first location is located on a first side of the shuttle and wherein the second location is located on a second side of the shuttle that is opposite the first side.

8. The assembly of claim 5, wherein the plurality of permanent magnets includes neodymium magnets, ceramic magnets, samarium cobalt magnets, or any combination thereof.

9. The assembly of claim 1, wherein the shuttle includes a ferromagnetic material and has an absence of a permanent magnet.

10 The assembly of claim 1, wherein the shuttle is configured to rotate about the longitudinal axis of the delivery device.

11. The assembly of claim 1, wherein the aperture is an elongate aperture.

12. The assembly of claim 11, wherein the elongate aperture is a linear elongate aperture.

13. The assembly of claim 12, wherein the linear elongate aperture is a linear elongate slot or a linear elongate cylinder.

14. The assembly of claim 1, wherein the shuttle includes radial fins.

15. The assembly of claim 14, wherein:
the delivery device is a syringe including a barrel, a plunger, and a stopper, wherein the barrel is configured as the reservoir; and
the syringe is a preloaded syringe that includes the hydrogel.

16. An injectable hydrogel agitation and delivery system, the system comprising:
a syringe including a barrel, a plunger, and a stopper, wherein the barrel is configured as a reservoir for an injectable hydrogel;
a shuttle disposed in the reservoir, wherein at least a portion of an outer surface of the shuttle is engaged with an inner surface of the barrel, and wherein the shuttle is configured to rotate about and translate along a longitudinal axis of the syringe to agitate an injectable hydrogel in the reservoir responsive to an application of a magnetic field to the shuttle, and wherein the shuttle includes an aperture extending entirely through the shuttle along the longitudinal axis of the syringe; and
a magnetic activator including a magnet configured to emit the magnetic field to the shuttle.

17. The system of claim 16, wherein the magnetic activator includes an arcuate region configured to mechanically couple to an outer surface of the barrel.

18. The system of claim 17, wherein the magnetic activator includes:
a first end region extending from a first end of the arcuate region, wherein the first end region includes a first magnet disposed therein; and
a second end region extending from a second end of the arcuate region, wherein the second end region includes a second magnet disposed therein.

19. A kit comprising:
an injectable hydrogel comprising one or more types of polymeric hydrogen bond donors, one or more types of polymeric hydrogen bond acceptors, and water;
a syringe including a barrel, a plunger, and a stopper, wherein the barrel is configured as the reservoir for the injectable hydrogel;
a shuttle disposed in the reservoir, wherein the shuttle is configured to rotate about and translate along a longitudinal axis of the syringe responsive to an application of a magnetic field to the shuttle, and wherein the shuttle includes an aperture extending entirely through the shuttle along the longitudinal axis of the syringe; and
a magnetic activator including a magnet configured to emit the magnetic field to the shuttle, wherein the magnetic activator is configured to mechanically couple to an outer surface of the barrel of the syringe.

20. The kit of claim 19, wherein the syringe further comprises a preloaded syringe including the injectable hydrogel disposed in the reservoir.
